(19) United States
(12) Patent Application Publication
(10) Pub. No.: US 2008/0065002 A1
(43) Pub. Date: Mar. 13, 2008

(54) Catheter for Localized Drug Delivery and/or Electrical Stimulation

(75) Inventors: Thomas J. Lobl, Valencia, CA (US); John V. Schloss, Saugus, CA (US); Anna Imola Nagy, Valencia, CA (US); Jacob E. Pananen, Pasadena, CA (US)

Correspondence Address: BANNER & WITCOFF, LTD., 1100 13th STREET, N.W., SUITE 1200, WASHINGTON, DC, 20005-4051, US

(73) Assignee: NEUROSYSTEC CORPORATION, Valencia, CA (US)

(21) Appl. No.: 11/850,156

(22) Filed: Sep. 5, 2007

Related U.S. Application Data
(60) Provisional application No. 60/824,895, filed on Sep. 7, 2006.

(51) Int. Cl. A61N 1/30

(52) U.S. Cl. 604/21

(57) ABSTRACT

A drug-compatible, biocompatible, drug-delivery catheter can include multi-lumen tubing attached to an end fitting, with the end fitting having an internal fluid chamber and a fluid exit region. The catheter can also include multi-lumen tubing having one or more needles attached at a distal end.

### CROSS-REFERENCE TO RELATED APPLICATION

This application claims the benefit of U.S. Provisional Application Ser. No. 60/824,895, filed Sep. 7, 2006 and titled “Catheter for Localized Drug Delivery and Electrical Stimulation,” hereby incorporated by reference herein.

### BACKGROUND

Delivery of drugs to specific tissue locations can be accomplished using a catheter system. As but one example, a catheter system can be used to deliver a tissue-specific drug to the middle ear or to the inner ear (e.g., to the cochlea). However, materials used in existing catheters can bind to drugs being delivered by a catheter, thereby reducing the actual concentration of delivered drug below an expected level. In particular, many therapeutic compounds are small organic molecules with solubility in organic solvents and much less solubility in aqueous media. These therapeutics frequently have a high affinity for plastic surfaces and often even dissolve into the plastic materials used to fabricate drug delivery devices. In some cases, the drug can actually pass through plastic catheter walls and into the patient at an undesired location. When this happens, the drug concentration within the liquid phase inside the catheter is reduced and the patient does not receive the desired amount of drug at the correct location. Many existing catheter materials are also permeable to water and other solutes. Such permeability can also cause drugs delivered in low volumes and at slow flow rates to have their concentrations unpredictably altered.

Another complicating factor is the thrombogenicity of materials used in existing catheter designs. Thrombogenic catheter materials used in drug-delivery systems may foster the development of blood clots and other kinds of fibrous clots that block drug delivery through holes, pores, screens or membranes. This can prevent effective drug delivery and can promote stenosis.

Apart from problems associated with drug absorption/adsorption and permeability of catheter materials, targeted delivery of drugs to various confined spaces (e.g., the middle or inner ear) presents additional challenges. In some treatments, it is useful to simultaneously deliver a drug-laden liquid to a region and provide a path for excess liquid to escape. Known existing devices and techniques for such simultaneous delivery and escape have proved less than completely satisfactory.

### SUMMARY

This Summary is provided to introduce a selection of concepts in a simplified form that are further described below in the Detailed Description. This Summary is not intended to identify key features or essential features of the invention.

In some embodiments, a drug delivery system is fabricated from materials that will have low affinity for various drug substances. In some such embodiments, the drug delivery system includes a catheter having a multi-lumen tube and an end fitting, with lumens of the tube flowing into (or out of) a chamber inside of the end fitting. Various types of end fittings can be employed. In certain embodiments, electrodes are located on (or in) the end fitting and/or on a portion of the multi-lumen tube to which the end fitting is attached. Such electrodes, when coupled to an appropriate electronics package, permit electrical stimulation of an ear region or other tissue and/or electrically-driven drug delivery. In some additional embodiments, a catheter includes a multi-lumen tube having needles at the distal end, with each needle having a passage in fluid communication with a lumen of the tube.

Catheters according to these and other embodiments can be used to deliver a variety of drugs to a variety of different bodily regions. In some embodiments, a catheter end fitting is configured for placement in the round window niche. In other embodiments a catheter and/or an end fitting is configured for placement elsewhere in the body.

### BRIEF DESCRIPTION OF THE DRAWINGS

The foregoing summary and the following detailed description are better understood when read in conjunction with the accompanying drawings, which are included by way of example, and not by way of limitation.

FIG. 1 shows a distal end of a catheter according to at least some embodiments.

FIG. 2 is a longitudinal cross-sectional view of the distal end of the catheter of FIG. 1.

FIG. 3 shows a distal end of a catheter, according to at least some embodiments, having an end fitting that is curved to facilitate more convenient placement into a round window niche.

FIG. 4 shows a distal end of a catheter, according to at least some embodiments, having a flared end fitting and a flat front.

FIG. 5 is a longitudinal cross-sectional view of the distal end of the catheter of FIG. 4.

FIG. 6 shows a distal end of a catheter, according to at least some embodiments, having a flared end fitting and a meshed screen on the face configured for placement adjacent to the round window membrane.

FIG. 7 shows a distal end of a catheter, according to at least some embodiments, having a cylindrical end fitting.

FIG. 8 is a longitudinal cross-sectional view of the distal end of the catheter of FIG. 7.

FIG. 9 shows a distal end of a catheter, according to at least some embodiments, having a cylindrical end fitting and an inflatable bladder.

FIG. 10 is a longitudinal cross-sectional view of the distal end of the catheter of FIG. 9.

FIG. 11 shows a distal end of a catheter, according to at least some embodiments, having a flared end fitting and an inflatable bladder.

FIG. 12 shows a distal end of a catheter, according to at least some embodiments, having suture anchors.

FIG. 13 shows a distal end of a catheter, according to at least some embodiments, having an electrode embedded into the side wall of a bulb end fitting.

FIG. 14 is a longitudinal cross-sectional view of the distal end of the catheter of FIG. 13.

FIG. 15 shows a distal end of a catheter, according to at least some embodiments, having active and ground electrodes embedded in the side wall of a bulb end fitting.

FIG. 16 is a longitudinal cross-sectional view of the distal end of the catheter of FIG. 15.

FIG. 17A shows a distal end of a catheter, according to at least some embodiments, having an active electrode on an outer surface of a bulb end fitting and a ground electrode on an outer surface of the catheter tube away from the end fitting.

FIG. 17B is a cross-sectional view from the location shown in FIG. 17A.

FIG. 17C shows a distal end of a catheter, according to at least some embodiments, having an active electrode inside of a bulb end fitting and a ground electrode on an outer surface of the catheter tube away from the end fitting.

FIG. 17D shows the distal end of the catheter of FIG. 17C with the bulb end fitting removed.

FIG. 18 shows a catheter, according to various embodiments, having a delivery tube, an extraction tube, an electrode wire and an electronics package at a proximal end.

FIGS. 19-22 show a distal end of a catheter, according to at least some embodiments, having a self-expanding member.

FIG. 23 shows a distal end of a catheter having two needles at the distal end.

FIGS. 24 and 25 show a distal end of a catheter having two needles at the distal end, with the needles configured to deliver electrical stimulation.

### DETAILED DESCRIPTION

The round window membrane separating the middle and inner ear is permeable to many drugs. Drugs delivered to the round window will diffuse through the membrane and reach the inner tissues. Catheters according to certain embodiments are designed to transfer fluids into and out of the inner ear through the round window membrane and are useful for delivering drugs to treat inner (and middle) ear conditions. Notably, therapeutics can be delivered on a temporary basis to the middle ear and/or to the round window niche to treat disease (e.g., an infection), to treat injury, or for other therapeutic purposes. For example, catheters according to certain embodiments can be used to treat tinnitus or sudden sensorineural hearing loss, as well as for the administration of neuroprotective drugs following acoustic trauma. Diagnostic drugs can also be delivered to a specific location so as to allow a physician to determine if a particular therapy will be helpful. Additional examples of ear and hearing-related conditions that can be treated with (or as part of) various embodiments are described below. The invention is not limited to use for treatment of conditions specifically identified, however.

Indeed, embodiments of the invention can be used for treatment of conditions affecting regions of the human body other than the middle or inner ear. Although the following description will in many instances refer to placement of components in a round window niche, this is only for purposes of illustration. Additional embodiments include devices such as are described below for round window drug delivery, but that have components sized or otherwise configured for placement into other body regions. Such other body regions include, but are not limited to, an auditory nerve, an optic nerve, an eye, a pituitary gland, an adrenal gland, a thymus gland, an ovary, a testis, a heart, a pancreas, a liver, a spleen, a brain (surface or implanted) or a spinal cord.

In addition to the devices described herein, further embodiments include use of these and other devices for delivery of drugs and/or electrical stimulation to treat any of various conditions.

Examples of drugs that can be used in (or in conjunction with) various embodiments include, but are not limited to, antibiotics (e.g., an aminoglycoside, an ansamycin, a carbacephem, a carbapenum, a cephalosporin, a glycopeptide, a macrolide, a monobactam, a penicillin), anti-viral drugs (e.g., an antisense inhibitor, a ribozyme, fomiversen, lamivudine, pleconaril, amantadine, rimantadine, an anti-idotype antibody, a nucleoside analog), anti-inflammatory steroids (e.g., dexamethasone, triamcinolone acetonide, methyl prednisolone), a neurologically active drug (e.g., ketamine, caroverine, gacyclidine, memantine, lidocaine, traxoprodil, an NMDA receptor antagonist, a calcium channel blocker, a GABA_{A} agonist, an α2δ agonist, a cholinergic, an anticholinergic), anti-cancer drugs (e.g., abarelix, aldesleukin, alemtuzamab, alitretinoin, allopurinol, altretamine, amifostine, anastrolzole, azacitidine, bevacuzimab, bleomycin, bortezomib, busulfan, capecitabine, carboplatin, carmustine, cisplatin, cyclophosphamide, darbepoetin, daunorubicin, docetaxel, doxorubicine, epirubicin, epoetin, etoposide, fluorouracil, gemicitabine, hydroxyurea, idarubicin, imatinib, interferon, letrozole, methotrexate, mitomycin C, oxaliplatin, paclitaxel, tamoxifen, topothecan, vinblastine, vincristine, zoledronate), or a fungicide (e.g., azaconazole, a benzimidazole, captafol, diclobutrazol, etaconazole, kasugamycin, or metiram). Analogs of the above-identified specific drugs (and other drugs) could also be used.

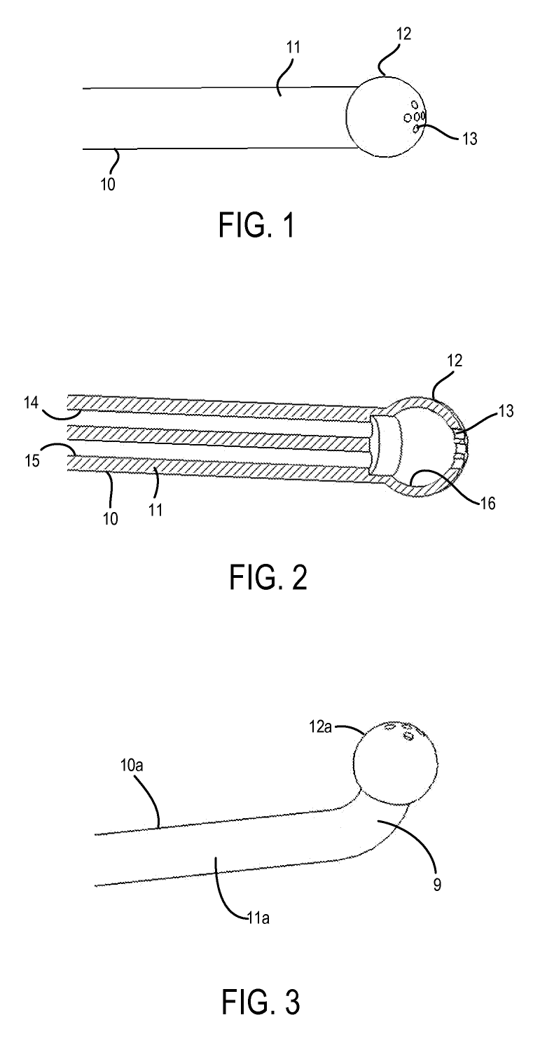

FIG. 1 shows the distal end of a catheter 10, according to one embodiment, that is configured for placement into (and for delivery of drugs via) the round window niche. Catheter 10 includes a length of multi-lumen tubing 11 attached to a bulb 12. FIG. 2 is a cross-sectional view of the distal end of catheter 10 and shows lumens 14 and 15 of tubing 11. Lumens 14 and 15 are both open to a chamber 16 within bulb 12. In use, bulb 12 is placed into a round window niche. Bulb 12 is sized to fit snugly in the round window niche. At the proximal end of catheter 10 (not shown), inflow lumen 14 would be connected (directly or via other intermediate components) to a source of drug-laden fluid (e.g., a port in fluid communication with an external pump or other source, an implanted pump). The drug-laden fluid would then flow through inflow lumen 14 into chamber 16. Fluid in chamber 16 would then exit through outlet holes 13 for delivery to the round window membrane. Excess fluid in chamber 16 is allowed to escape via outlet lumen 15. Lumen 15 can be connected to a valve (not shown) or other component that may be used to adjust the fluid pressure within chamber 16. Although tubing 11 of catheter 10 is a dual lumen catheter, other embodiments employ multi-lumen tubing having three or more lumens. FIG. 3 shows a distal end of a catheter 10a according to another embodiment. Catheter 10a is generally similar to catheter 10 of FIGS. 1 and 2, but includes curved fitting 9 (at the distal end of multi-lumen tubing 11a) that facilitates convenient placement of bulb 12a into a round window niche.

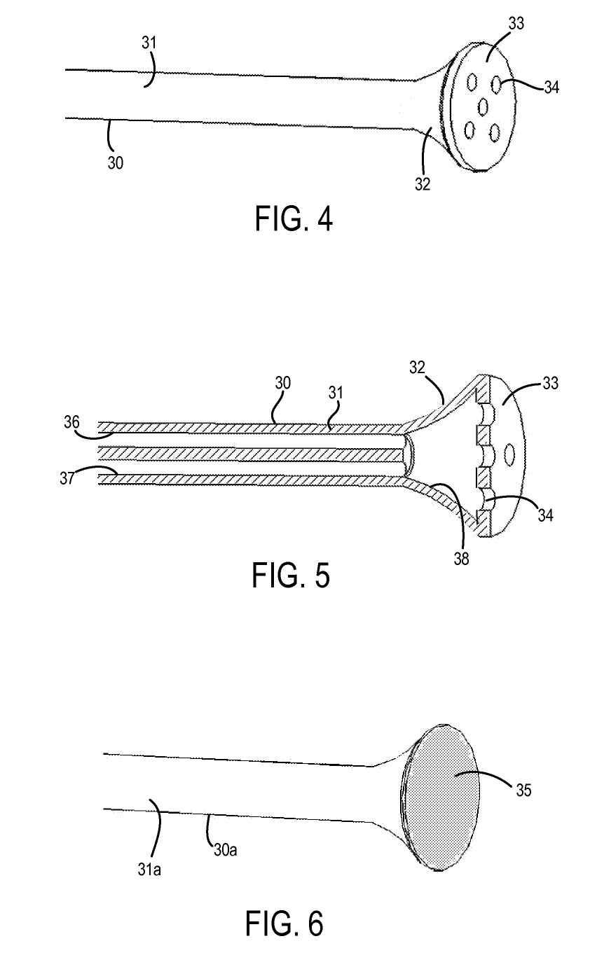

FIG. 4 shows a distal end of a catheter 30 according to another embodiment. Catheter 30 includes a length of multi-lumen tubing 31 attached to a flared end fitting 32. End fitting 32, which is also sized for placement in a round window niche, includes a flat front 33 to facilitate placement closer to the round window. Outlet holes 34 then deliver drug-laden fluid to the round window. As seen in FIG. 5, a cross-sectional view of the distal end of catheter 30, inflow lumen 36 and outflow lumen 37 both open into fluid chamber 38. Similar to catheter 10 of FIGS. 1 and 2, inflow lumen 36 can be connected to a source of drug-laden fluid, which flows from that source into chamber 38. Fluid in chamber 38 would then exit through outlet holes 34 for delivery to the tissue being treated. Excess fluid in chamber 38 is allowed to escape via outlet lumen 37. Outlet lumen 37 could similarly be connected to a valve or other component for controlling fluid pressure within chamber 38. As with catheter 10 of FIGS. 1 and 2, variations on catheter 30 include tubing with three or more lumens and/or curved end fittings. Yet another variation is seen in FIG. 6 as catheter 30a. Catheter 30a is generally similar to catheter 30 of FIGS. 4 and 5, but includes a different type of fluid exit region. Specifically, catheter 30a employs a meshed screen 35 instead of outlet holes 34 to transfer fluid from within the catheter to the round window membrane. In still other variations a permeable membrane could be used instead of meshed screen 35.

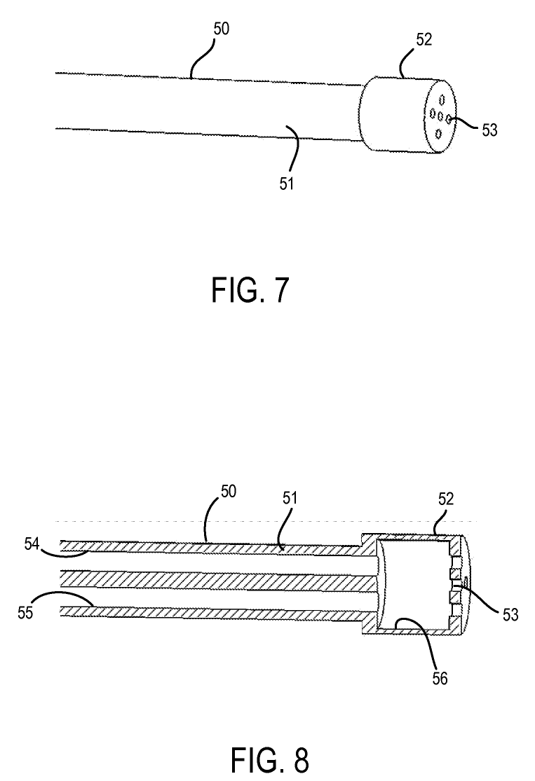

FIG. 7 shows the distal end of a catheter 50 according to a further embodiment. Catheter 50 includes a length of multi-lumen tubing 51 attached to a cylindrical tip 52. Tip 52, which is also sized for placement in a round window niche, also includes a flat front to facilitate placement closer to the round window. Outlet holes 53 deliver drug-laden fluid to the round window. As seen in FIG. 8, a cross-sectional view of the distal end of catheter 50, inflow lumen 54 and outflow lumen 55 open into fluid chamber 56. Similar to catheters 10 and 30, inflow lumen 54 can be connected to a source of drug-laden fluid, which flows from that source into chamber 56. Fluid in chamber 56 would then exit through outlet holes 53 for delivery to the tissue being treated. Excess fluid in chamber 56 is allowed to escape via outlet lumen 55, which could similarly be connected to a valve or other component for controlling fluid pressure within chamber 56. Variations on catheter 50 include the variations discussed above for catheters 10 and 30 (e.g., curved end fittings, tubing with three or more lumens, a meshed screen or permeable membrane).

The catheter end fittings in the embodiments of FIGS. 1-8 are designed to fit snugly in the round window niche. These designs also reduce the exposure of the fluid exit region of the end fitting (e.g., holes or screen) to bodily fluids to reduce clogging of the fluid exit region with blood or fibrin clots or other large particles. The end fitting of the catheter may be manufactured separately and bonded to the catheter tubing using epoxy or other known adhesives. The diameter of the end fitting in some embodiments may range from 1 mm to 4 mm (e.g., a diameter of 1.5 to 2.5 mm).

Catheters of the embodiments shown in FIGS. 1-8, as well in other embodiments described herein, may be made with drug- and bio-compatible fluoropolymers for better drug compatibility. Catheters prepared from fluoropolymers will have fewer (or no) drug incompatibility problems and provide improvement over conventionally-used materials.

Fluoropolymers, in particular polytetrafluoroethylene (PTFE), do not exhibit high affinity for hydrophobic drugs such as gacyclidine. PTFE is not thrombogenic and will not promote stenosis (the narrowing of a cavity, such as the auditory canal). Catheters fabricated from fluoropolymers thus will have advantages over catheters fabricated from other materials. In particular, drug delivery will be more efficient due to lower binding of hydrophobic drugs with the catheter, less diffusion of drug through catheter walls, less potential for occlusion by blood clots, and less potential for stenosis. Fluoropolymers that can be used in catheters in at least some embodiments include PTFE, hexafluoropropylene (HFP), tetrafluoroethylene (TFE), fluorinated ethylene-propylene (FEP, a copolymer of TFE and HFP), perfluoroalkoxy polymers (PFA, a copolymer of TFE and PPVE), ethylene tetrafluoroethylene (ETFE, a copolymer of TFE and ethylene), MFA (a copolymer of TFE and perfluoromethylvinyl ether (PMVE)), polychlorotrifluoroethylene (PCTFE), polyvinylidene difluoride (PVDF), polyvinyl fluoride (PVF), ethylene chloro-trifluoroethylene (ECTFE), THV (terpolymer of TFE, HFP and vinylidene fluoride (VF2)) and other known fluoropolymers (as listed by, e.g., J. George Drobny in Technology of Fluoropolymers, pages 1-3 (CRC Press, Boca Raton 2001)).

In some embodiments, the tubing and catheter end fitting are formed entirely from one or more fluoropolymers. In other embodiments, the tubing, the end fitting, and/or other components of the catheter may be formed from non-fluoropolymer materials and then coated or coextruded so that fluid-contacting regions (e.g., inner surfaces of lumens and of the fluid chamber) are covered with a fluoropolymer to maintain a low affinity for drug substances. Fabrication of a bulb (or other end fitting) from a fluoropolymer (or other biocompatible and drug compatible polymer) may also help prevent blood clot attachment to the end fitting.

As seen in FIG. 6, some embodiments include a flat region formed of a porous material (e.g., meshed screen or semipermeable membrane). In some embodiments, a larger part of a ball, conical, cylindrical or other shaped end fitting is made from such a porous material. As in other embodiments, that porous material may be a biocompatible and drug compatible material (e.g., a fluoropolymer), and may flexible and soft so as to permit easy insertion into the round window niche. The entire end fitting (whatever the shape) may be formed of porous material, or the end fitting may include porous and non-porous regions. In some embodiments, the end fitting may be the open end of the catheter with (or without) a porous screen of other material placed over the open end.

In some embodiments the end fitting can be squeezed with tweezers or forceps during implantation to make the insertion into the round window niche easier. As indicated above, and for embodiments designed for delivery of drugs to the round window membrane, the size of the end fitting is designed to comfortably fit in the round window niche. After implantation the squeezed end fitting will return to the original form and fit tightly in the round window niche. In other embodiments the end fitting is hard and not easily compressed with tweezers. In this case the catheter placement in the round window niche can be directed with tweezers holding the assembly on the neck just behind the end fitting and placing the hard end fitting in the correct position.

In some embodiments catheters are similar to those described above, but are configured for placement of the end fitting into a different anatomical region. In such embodiments, the end-fitting is appropriately sized based on the desired use of the catheter. In yet other embodiments, the end fitting of the catheter is removable, allowing a physician to replace it with an end fitting better suited for a particular therapy.

As indicated above, drugs can be actively released from an end fitting as part of a mobile phase flow from a pump or other device supplying a drug-laden liquid. In some cases, drugs exit the catheter passively (with or without fluid flow) through holes in the end fitting or by diffusion through a porous membrane in the end fitting. In particular, the chamber in the end fitting is filled with drug-laden fluid, but the fluid source does not actively pump additional fluid into the chamber and the outflow lumen is closed (e.g., via a valve). A combined approach can also be used (i.e., passive drug delivery can be employed when an active device such as a pump is turned off or removed).

Liquid used for delivery of drug through the catheter can be supplied in various ways.

Examples include a syringe, a syringe pump, a mechanical pump, an osmotic pump, a MEMS pump or a piezoelectric pump. The delivery liquid can be, e.g., a homogeneous liquid-drug formulation, a particulate suspension containing drug (e.g., a nanoparticulate formulation), or a liquid passing through a solid drug eluting component, as described in any of commonly-owned U.S. patent applications Ser. No. 11/414,543 (titled “Apparatus and Method for Delivery of Therapeutic and Other Types of Agents” and filed May 1, 2006), Ser. No. 11/759,387 (titled “Flow-Induced Delivery from a Drug Mass” and filed Jun. 7, 2007), Ser. No. 11/780,853 (titled “Devices, Systems and Methods for Ophthalmic Drug Delivery” and filed Jul. 20, 2007) and/or Ser. No. 11/831,230 (titled “Nanoparticle Drug Formulations” and filed Jul. 31, 2007), all of which applications are incorporated by reference herein. As indicated above, a return flow path away from the treated region (e.g., the outflow lumens in FIGS. 2, 5 and 8) can be connected to a valve for opening and closing. The outflow lumen could also (or alternatively) be connected to a receiving reservoir, connected to a pressure sensor equipped with a pressure regulating outlet valve, etc. In many cases, an outlet pressure sensor or an outlet valve may not be needed. If the delivery flow is regulated by use of an outlet valve, then the outlet pressure can be maintained by cycling the valve between the open and closed positions. The outlet pressure or the inlet back pressure can also be used to shut down a supply pump and alert a physician by way of an integrated alarm if the drug delivery system becomes clogged or otherwise resistant to flow.

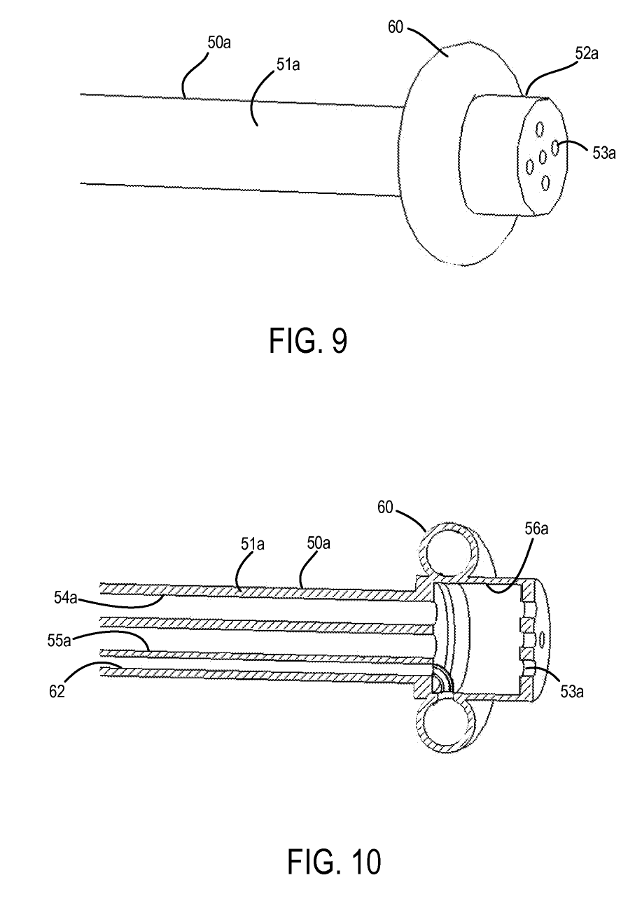

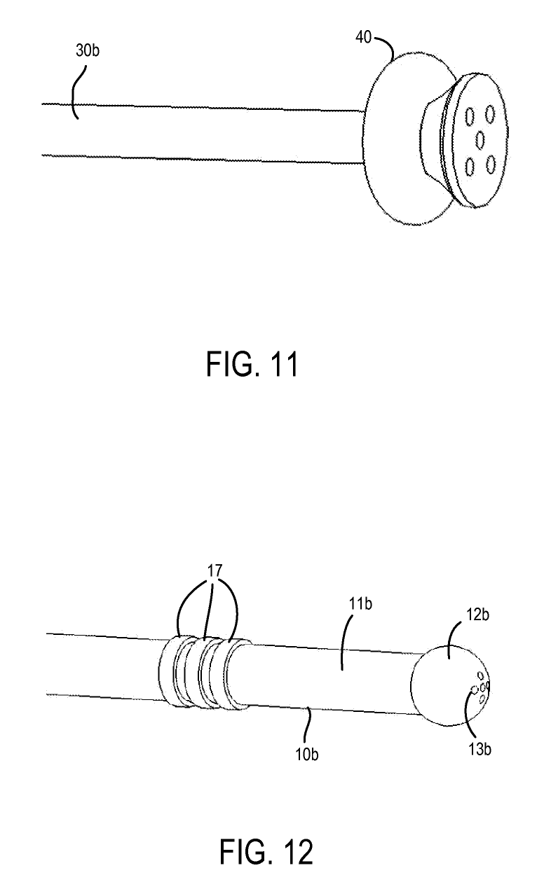

FIG. 9 shows the distal end of a catheter 50a according to another embodiment. Catheter 50a includes a multi-lumen tube 51a and a cylindrical tip 52a having outlet holes 53a in a flat face. Catheter 50a is similar to catheter 50 of FIGS. 7-8, but includes an inflatable bladder 60. In use, end fitting 52a is placed into the round window niche. Bladder 60 then inflates to engage the internal side wall of the round window niche, secure catheter 50a and end fitting 52a in place, and provide a fluid seal between the niche and the rest of the middle ear. FIG. 10 is a cross-sectional view of catheter 50a showing inflow lumen 54a and outflow lumen 55a opening into fluid chamber 56a. Tubing 51a includes an additional lumen 62 through which an inflation fluid (preferably a gas such as air, nitrogen or oxygen or a liquid such as water) can be delivered into bladder 60. FIG. 11 shows a catheter 30b according to a further embodiment. Catheter 30b is generally similar to catheter 30 of FIGS. 4 and 5, but includes an inflatable bladder 40 which operates similar to bladder 60 of FIGS. 9 and 10.

In some embodiments the catheter tubing may include suture anchoring elements. FIG. 12 shows a catheter 10b according to one such embodiment. Catheter 10b is similar to catheter 10 of FIGS. 1 and 2, but has suture anchors 17 along a portion of tubing 11b that provide a method for securing the distal end of catheter 10b (and thus, end fitting 12b) in place. Sutures may be used to attach the catheter to tissue in the middle ear or in the auditory canal, or externally to the ear, to prevent the catheter tip from slipping out of the round window niche. Although suture anchors 17 in FIG. 12 are ring-shaped, other shapes (e.g., squares, half-rings, thin plates or “ears” with holes for suture thread) can be employed. In some embodiments, suture anchors may consist of larger diameter rings cut from polymer tubes and attached to the catheter using epoxy, other kinds of glue, or adhesives. In still other embodiments, suture anchors may be manufactured as part of the extrusion process or they may be heat-formed. Alternatively, suture anchors may be bumps on the surface of the tubing made of silicone elastomer, epoxy, or other kinds of adhesives. The number and location of suture anchoring elements may vary, but preferably there is a set of suture anchors 3 cm from the distal tip of the catheter.

In addition to delivery of drugs, catheters according to some embodiments include electrodes to provide electrical stimulation to tissue. For example, electrical stimulation of the cochlear round window, the promontory, or the external ear has been known to suppress tinnitus in some patients. As with embodiments in which the catheter is only used for drug delivery, a combined electrical stimulation/drug delivery catheter system can be implanted in the round window niche positioned towards the round window. One or more electrodes in (or near) the end fitting can be coupled to an electronics package and used to stimulate the nerves of the cochlea, the nerves running through and near the middle ear, the round window and/or the promontory area adjacent to the round window. The electrode(s) are designed to deliver a reliable electrical charge/potential as directed by the electronics package. In some embodiments, one or more electrodes is on the catheter end fitting and a ground electrode is placed where it is needed. The electronics package may be placed external to the patient's middle ear and the auditory canal for convenience (e.g., behind the ear or as part of the pumping system). The electronics can be battery operated and have an on/off switch, or can be powered via radio frequency transmission or use some other wireless electronic stimulator which will not require a battery.

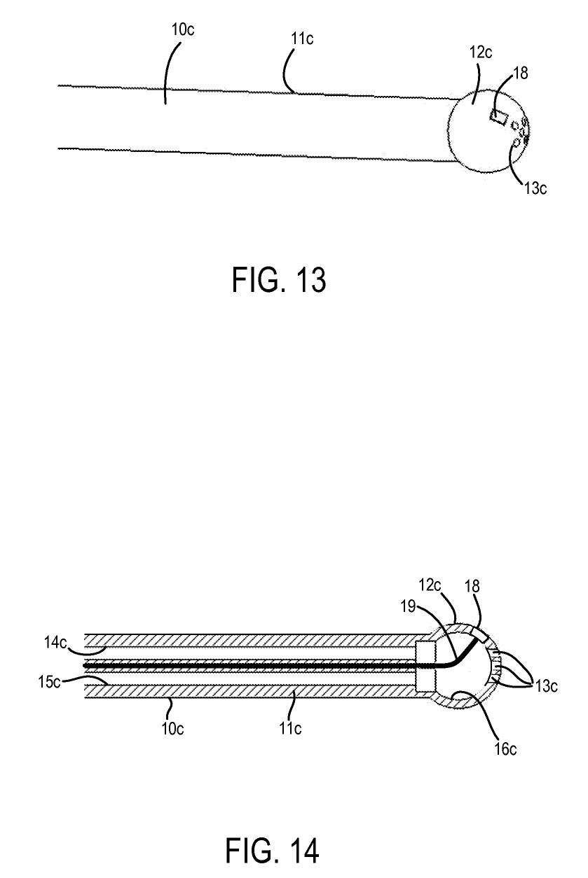

FIG. 13 shows the distal end of a catheter 10c according to one embodiment of a combined drug delivery and electrical stimulation catheter. Catheter 10c includes a multi-lumen tube 11c and a bulb-shaped end fitting 12c having outlet holes 13c. Catheter 10c is similar to catheter 10 of FIGS. 1 and 2, but further employs an electrical potential transmission system to deliver electrical potentials to the middle/inner ear, via the round window membrane, from electrode 18. FIG. 14 is a cross-sectional view of the distal end of catheter 10c and shows inflow lumen 14c, outflow lumen 15c and fluid chamber 16c. Electrode wire 19 is connected to electrode 18. In some embodiments, tubing 11c may contain an additional lumen through which wire 19 runs (from a voltage generator) to electrode 18. In other embodiments wire 19 may be molded within the side wall of tubing 11c during manufacturing, or may be attached to the outer surface of the side wall using a conventional adhesive composition. In still other embodiments, wire 19 (or multiple wires, in the case of some embodiments such as are described below) may simply pass through one of the existing lumens in tubing 11c (e.g. one of inflow lumen 14c or outflow lumen 15c), or wire 19 may be coextruded with tubing 11c during manufacturing. The tip of electrode 18 may be bonded to the terminal end of catheter 10c or molded or laminated inside the catheter. The electrode tip may encompass a variety of different forms. In at least some embodiments, the surface of the electrode that will contact the patient's tissue is planar or near-planar. A spherical electrode could also be used. The electrode on the catheter may be manufactured of the same material used to construct the electrode wire. The wire may be constructed of titanium, platinum or other biocompatible, drug compatible, conductive materials, and from alloys such as Nitinol (55% nickel, 45% titanium alloy), titanium 6,4 (Ti6Al4V) or platinum-iridium.

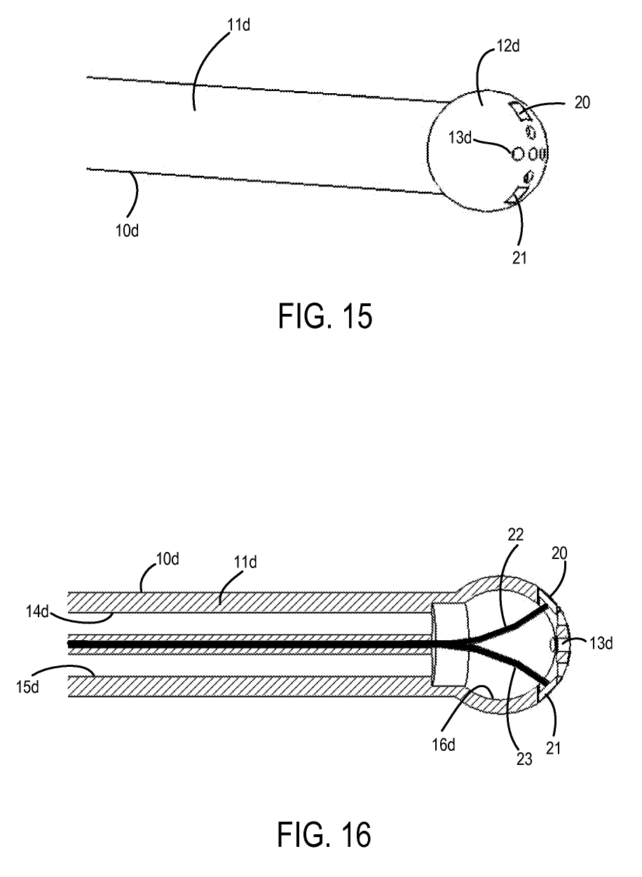

FIGS. 13 and 14 illustrate a single electrode embedded in the side wall of a catheter tip. FIG. 15 shows a distal end of a catheter 10d according to another embodiment. Catheter 10d is similar to catheter 10c, but includes a catheter tip with two adjacent electrodes. In particular, bulb end fitting 12d is attached to multi-lumen tubing 11d and includes an active electrode 20 and a return (ground) electrode 21 adjacent outflow holes 13d. FIG. 16 is a cross-sectional view of the distal end of catheter 10d and shows inflow lumen 14d, outflow lumen 15d and chamber 16d. Electrical wires 22 and 23 connect to electrodes 20 and 21. Wires 22 and 23 can be incorporated into and/or attached to catheter 10d in any of the manners described above for catheter 10c (e.g., passed together through a single lumen), and electrodes 20 and 21 can have a variety of shapes and be formed from a variety of materials (as also described in connection with catheter 10c).

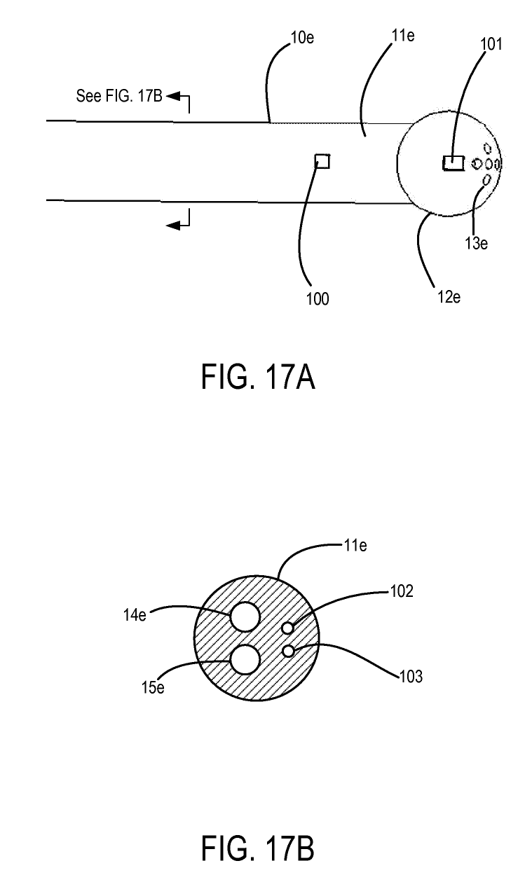

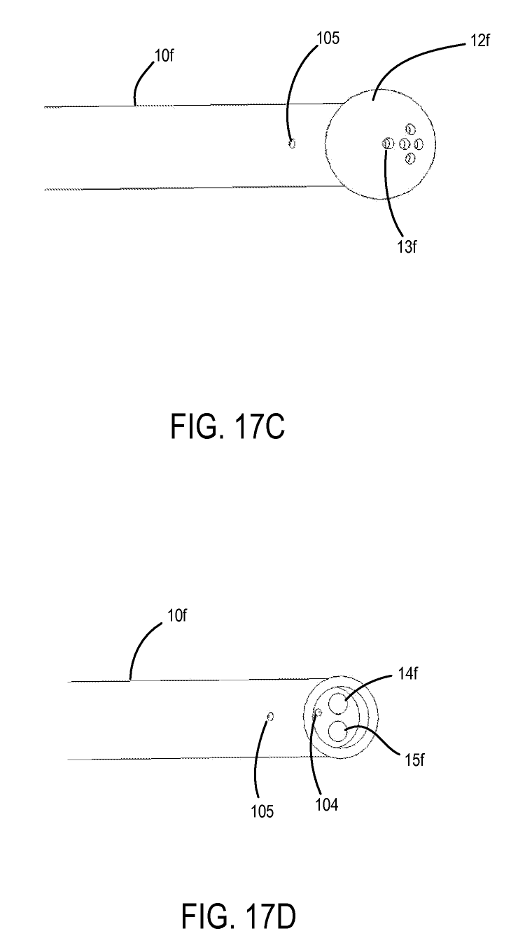

A ground electrode may be outside the ear, or may be in the middle ear away from the round window membrane. FIG. 17A shows a distal end of a catheter 10e according to an embodiment that is similar to catheter 10d, but in which a ground electrode 100 is located on a distal end portion of multi-lumen tubing 11e instead of on bulb end fitting 12e. FIG. 17B is a cross-sectional view from the location shown in FIG. 17B and shows inflow lumen 14e, outflow lumen 15e, electrode wire lumen 102 (through which a wire passes to one of electrodes 100 and 101) and electrode wire lumen 103 (through which another wire passes to the other of electrodes 100 and 101). In the embodiment of FIGS. 17A and 17B (as well as in other embodiments described herein), the active and/or ground wires may be separately insulated (e.g., with a fluoropolymer heat-shrink tubing) prior to passing those wires through a designated lumen in a catheter. Alternatively, wires may be threaded through and sealed within separate lumens of a multi-lumen catheter without additional coatings. In yet other variations, wires may be individually insulated and routed through a single lumen (e.g., two wires passed through one of lumens 102 or 103).

FIG. 17C shows a distal end of a catheter 10f according to another embodiment. Catheter 10f is similar to catheter 10e, but includes an active electrode located inside of catheter end fitting 12f. When implanted in the patient, the inside of bulb end fitting 12f is filled with a fluid medium which will allow for the movement of charged molecules when an electrical potential is applied to the electrodes. Ground electrode 105 may be located outside the round window niche on the catheter wall. A fluid seal between the active and ground electrode (created by an inflatable bladder such as described above or a self-expanding ring such as described below) could be included to force the electrical potential to be applied across the round window membrane and/or the promontory. Examples of fluid media (vehicles) that can be used in these and other embodiments include (but are not limited to) saline, artificial perilymph, Ringer's solution and lactated Ringer's solution. FIG. 17D shows the distal end of catheter 10f with bulb 12f removed to expose active electrode 104, inflow lumen 14f and outflow lumen 15f.

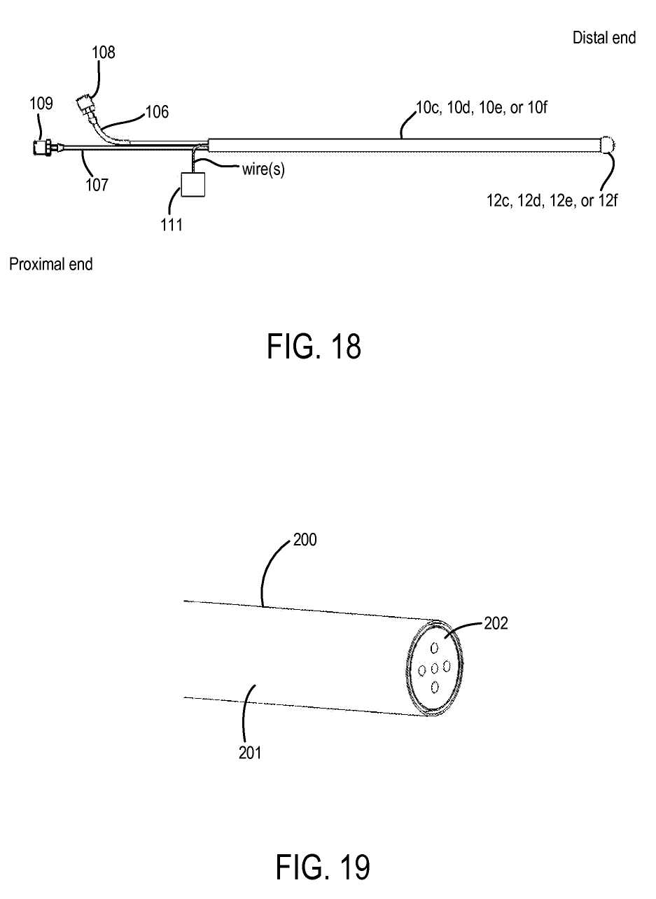

FIG. 18 illustrates the proximal and distal ends of a drug delivery and electrical stimulation device. Although FIG. 18 shows a catheter (e.g., one of catheters 10c, 10d, 10e or 10f) having a bulb shaped end fitting (e.g., one of end fittings 12c, 12d, 12e or 12f), the configuration of FIG. 18 could be used with catheters having other types of end fittings. Electrode wire(s) extend(s) from the proximal end of the catheter tubing and is (are) connected to an electronics package 111. Package 111 includes electronics that generate high frequency pulse trains. Those pulse trains are delivered (via the wire(s) and electrode(s)) to the middle/inner ear for the treatment of tinnitus and/or another condition. Electronics package 111 optionally includes a power supply (e.g., battery) and user interface (e.g., magnetically-activated on/off switch). A fluid delivery tube 106 and a fluid extraction tube 107 are respectively connected to the inflow and outflow lumens. Luer tips 108 and 109 at the proximal ends of tubes 106 and 107 allow for convenient attachment to a syringe, pump, valve, pressure gage, etc.

A combined electrical stimulation/drug delivery catheter can also be used with an implanted pump or port for tinnitus suppression or other treatments. Therapeutic fluid may be delivered via an osmotic pump or may be introduced through a subcutaneous port. Examples of such ports and pumps are described in the commonly-owned U.S. patent applications incorporated by reference above.

As discussed above, various embodiments include a bladder to provide a more secure fit of the end fitting in a round window niche. In other embodiments, an end fitting can include a collar in combination with (or as an alternative to) a bladder. As with a bladder, a collar can help to keep the end fitting (and thus, the catheter system) in place. Specifically, the collar will adhere to the osseus border of the round window niche and allow a more secure fit of the end fitting in the niche. In some embodiments, a collar is flexible and has a cylindrical shape and can be compressed during implantation with tweezers or forceps. After positioning in the round window niche, the compressed collar will return to the original shape, thereby providing frictional engagement with the wall of the round window niche. In some embodiments, the outer surface of the collar can include surface features (e.g., bumps) to help increase such frictional engagement. Materials for a collar can include flexible biocompatible materials such as silicone or polyurethane. In certain embodiments a collar includes a stent-like expandable ring around the catheter tip which will secure the catheter in the round window niche.

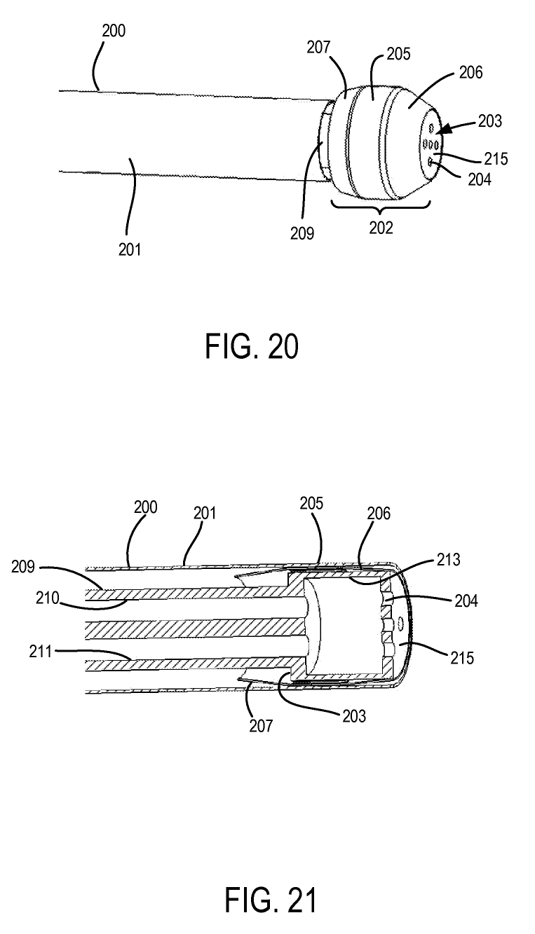

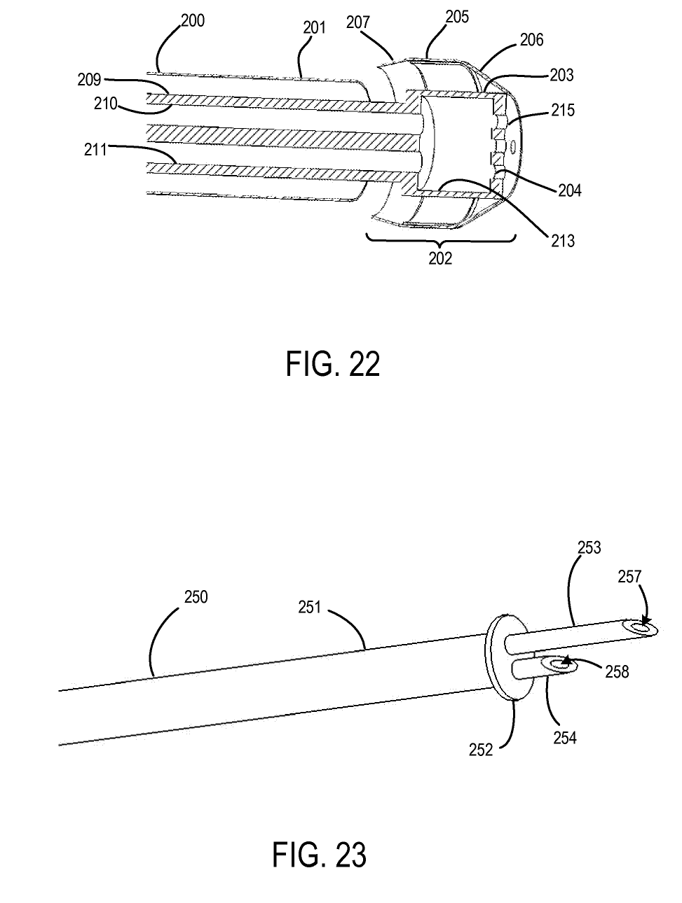

FIG. 19 shows the distal end of a catheter 200 that includes a stent-like expanding ring collar on an end fitting 202. In FIG. 19, end fitting 202 is withdrawn into the end of an outer sheath 201. In this configuration, the distal end of catheter 200 can be pushed by a physician into the round window niche. Once the distal end of catheter 200 is in its desired location, and as shown in FIG. 20, sheath 201 can be pulled back to force end fitting 202 out of the end of sheath 201. As end fitting 202 emerges from sheath 201, self-expanding ring 205 expands outward. Flexible polymer skirts 206 and 207 are coupled to ring 205 and also expand outward. Ring 205 and skirts 206 and 207 fit relatively tightly against the internal side wall of the round window niche to secure end fitting 202 in place and providing a relatively fluid-tight seal between the niche and the rest of the middle ear. FIG. 21 is a cross-sectional view of the distal end of catheter 200 when in the configuration of FIG. 19. As seen in FIG. 21, catheter 200 includes a dual lumen tube 209 (having inflow lumen 210 and outflow lumen 211), cylindrical end piece 203 (having outlet holes 204 on face 215), and an internal chamber 213. In use, catheter 200 operates in a manner similar to that described above for catheters of other embodiments. FIG. 22 is a cross-sectional view of the distal end of catheter 200 when in the configuration of FIG. 20. Skirt 206 is permanently attached to end piece 203 near edge of front face 215 using flexible, biocompatible adhesives, epoxy or other kinds of adhesives known in the art. Ring 205 is attached to skirts 206 and 207 such that, upon movement of end fitting 200 out of sheath 201, ring 205 expands to its original shape and pushes skirts 206 and 207 outward. For embodiments in which a relatively fluid tight seal is not required, skirts 206 and/or 207 may be omitted and ring 205 attached directly to end piece 203. Self-expanding ring 205 can be made of a material which exhibits shape-memory, e.g., Nitinol. An expanding collar can also be used in connection with embodiments providing electrical potential transmission or stimulation through electrodes. In some such embodiments, the expanding metal ring (such as ring 205) can be used as an electrode.

In still other embodiments not shown in the drawings, a skirt-type cover member is located at the end of a catheter to be positioned in the round window niche, and is used to form a fluid-receiving zone. The cover member is positioned above the round window membrane in the round window niche to form a fluid receiving zone adjacent to the round window membrane. Drug-containing fluid is delivered through the catheter and the cover member into the fluid receiving zone. The drug-laden fluid will pass through the round window membrane by diffusion, active transport or osmosis, thereby moving into the inner ear for treatment. Any remaining residual fluid in the fluid-receiving zone can be withdrawn from the patient. Extraction of the residual fluids is accomplished by applying light suction on a second end of a fluid extraction lumen. Alternatively, the device can be removed and the residual fluid will remain in the middle ear or be swallowed.

While catheters according to some embodiments release drug in the round window niche for diffusional passage through (or, in some selected cases, active transport across) the round window membrane, other embodiments can be used for injection of medications across the round window and into the cochlea. One such embodiment is shown in FIG. 23. Specifically, FIG. 23 shows the distal end of a catheter 250 having a dual lumen tube 251, a flexible insertion stop 252, and needles 253 and 254. If fluid is injected into the inner ear using only a single needle, a corresponding amount of perilymph must be displaced out of the cochlea (e.g., through the cochlear aqueduct). Accordingly, the rate of drug delivery with a single needle is limited by the tolerable increase in pressure that accompanies injection and the time required to reestablish normal pressure in the cochlea (3 to 15 seconds per injection). However, use of two needles divided by a partition, in conjunction with a two lumen catheter, allows for faster rates of injection without concomitant increase in intracochlear pressure. Needle 253 can be used to deliver medication across the round window membrane and needle 254 permits displacement of perilymph out of the cochlea. Needle 253 is in fluid communication with an inflow lumen of tubing 251 and needle 254 is in fluid communication with an outflow lumen of tubing 251. Insertion stop 251 may be clear, and is sized for placement within the round window niche. The fluid outlet 257 of needle 253 is further than the fluid outlet 258 of needle 254 from the end of tubing 251 (and more distally located) by, e.g., 0.1 to 1.5 mm. In some embodiments, two needles may be combined into a single structure (e.g., a dual lumen needle) having separate fluid passages, and with one of those fluid passages having an outlet that is more distally located than an outlet of the other of those fluid passages.

In some embodiments an outlet pressure sensor and pressure valve are coupled to the outflow lumen of tubing 251 so as to maintain physiologic intracochlear pressure (e.g., 0.5 to 1.5 mm Hg) independent of the rate of flow used for medication delivery. Alternatively, the outflow lumen can remain fully open, such that there is little or no pressure buildup during delivery of medication. The cochlear pressure can be maintained at the desirable level by appropriate use of the return pressure regulating outlet valve.

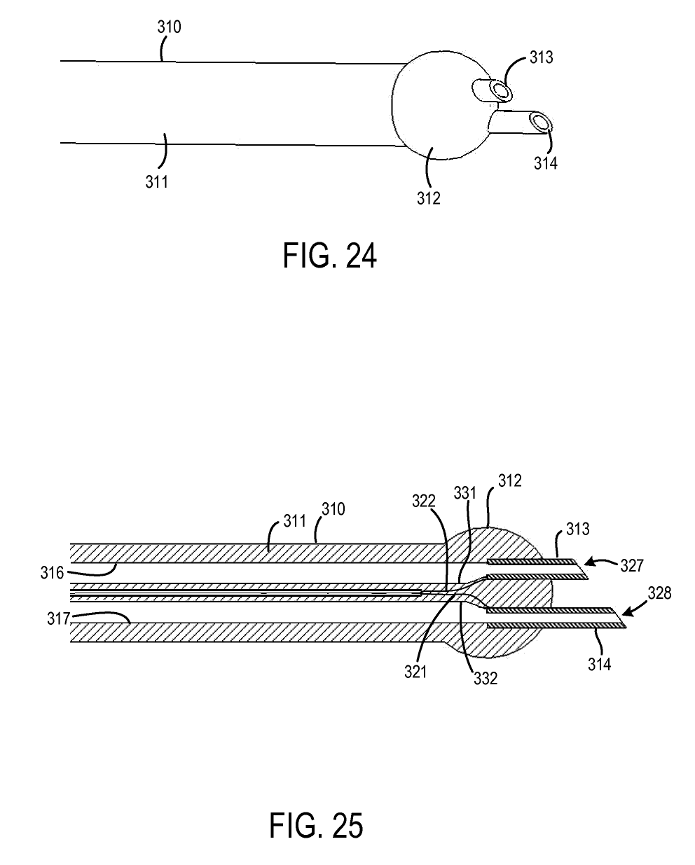

In some embodiments needle-equipped catheters such as catheter 250 may also include one or more electrodes for stimulation of the inner ear or promontory. In some such embodiments, such as catheter 310 shown in FIGS. 24 and 25, the needles are also used as electrodes. Catheter 310 includes a multi-lumen tube 311 with a bulb shaped end 312 attached to the distal end of tube 311. Needles 313 and 314 extend from end 312, with needle 314 extending further than needle 313. As seen in FIG. 25, the exposed opening 328 of the fluid passage in needle 314 is further from the bulb of end 312 (and more distally located) than the exposed opening 327 of the fluid passage in needle 313. Inflow lumen 317 is connected to the fluid passage of needle 314 by internal passage 332. Outflow lumen 316 is connected to the fluid passage of needle 313 by internal passage 331. Drug-laden fluid is delivered to the inner ear through needle 314, with excess fluid exiting the inner ear through needle 313. A wire 322 is connected to needle 313 and another wire 321 is connected to needle 314. Wires 321 and 322 are separately insulated and routed through another lumen of tubing 311. In some embodiments, needles 313 and 314 are formed from platinum or other material that does not erode in the presence of electrical current. Catheter 310 could be used, e.g., in the configuration shown in FIG. 18.

In at least some embodiments employing needles to pierce the round window membrane and deliver drugs, an antibacterial filter is employed to help ensure the sterility of drug-laden fluid delivered to the cochlea. Such an antibacterial filter can be located in any of various locations in the fluid path between a source of drug-laden fluid and an outlet of the catheter delivering drug to the cochlea.

For at least some treatments, it is known that the ionic composition and osmolality of medication in a liquid delivery vehicle should match that of perilymph. This can be of greater importance when two needles are employed to give faster infusion rates, resulting in more efficient exchange of intracochlear fluid. One example of a suitable vehicle is Ringer's solution at an osmolality of 290 to 310 mOsm. At the injection flow rates that can be accomplished with a single needle, distribution of drug in the cochlea is dominated by diffusion. However, at the higher infusion flow rates that are possible with two needles (a drug delivery needle and an outlet needle), delivery of drug to the cochlea can be achieved more rapidly by fluid exchange.

### Drug Compatibility of Different Tubina Types

Several experiments were performed to prove the advantages and drug compatibility of the fluoropolymers to be used in round window catheters according to at least some embodiments. The experiments were performed using a solution of gacyclidine (also known as GK11), a drug that is soluble in its acid form, water insoluble and lypophilic in its basic form. Its water soluble form has affinity for hydrophobic surfaces, such as would be formed by many polymers used to fabricate conventional catheters. As such, it serves as a model indicator and predictor for drug loss that might be encountered due to binding (adsorption and absorption) to surfaces of materials used in catheter fabrication.

### EXAMPLE 1

### Tube Soaking

The low adsorption/absorption characteristics of fluoropolymer tubing versus other materials is shown in Table 1. The following tubing materials were evaluated: PTFE (polytetrafluoroethylene), FEP (fluorinated-ethylene-propylene), PVC, trilaminar coaxial tubing and three different types of silicone tubing. Four pieces of each type of tubing material were cut into ½ inch lengths. All the pieces were washed using isopropyl alcohol. The pieces of tubing were then soaked for 20 hours at room temperature in vials containing 100 μM gacyclidine in Ringer's Lactate at pH 6.0. The tubing pieces were placed in glass sample vials, two pieces of tubing in each vial. One milliliter of 100 μM gacyclidine in Ringer's Lactate at pH 6.0 was placed in each vial. The concentration of gacyclidine was determined by spectrophotometry at 234 nm and by HPLC. The FEP and PTFE tubing pieces showed very low retention of gacyclidine. PVC demonstrated high retention with around 20% adsorbed and/or absorbed. The silicones had high adsorption and/or absorption ranging from 27 to 58%. Results for specific tubing pieces are shown in Table 1.

TABLE 1
(tube soaking loss)

| Material | % Loss |
|---|---|
| FEP | 0.05 |
| PTFE | 0.5 |
| Tygon (PVC) | 21 |
| Trilaminar coaxial tubing | 36 |
| (including HD polyethylene, | |
| copolyester/ether) | |
| Small silicone tubing type A | 27 |
| Small silicone tubing type B | 48 |
| Small silicone tubing type C | 58 |

### EXAMPLE 2

### Drug Compatibility Study with PTFE Tubing

The compatibility of gacyclidine in Ringer's Lactate (pH 6.0) in PTFE tubing at room temperature and at 37° C. was evaluated. Three sets of six segments each of PTFE tubing were used. Six samples were collected at each of three time intervals (6 hr, 23 hr and 72 hr). For each set of samples collected, three were incubated at ambient temperature and three were incubated at 37° C. A 16.5 ft. long segment of PTFE tubing (0.010″ ID, 0.018″ OD) was filled with 100 μM gacyclidine in Ringer's Lactate solution (pH 6.0) by use of a glass syringe. The two ends of the tubing were sealed with a paraffin wax vapor barrier. After incubating at room temperature or 37° C. for a specified time, the solution was pumped directly into a glass HPLC autosampler vial insert using an air-filled syringe. The PTFE tubing drug loss in 72 hours at room temperature was 1.3% and at 37° C. was 7.9%. These results were not corrected for decomposition. The expected amount of decomposition expected in 72 hours at 37° C. is 8.0%. As such, there is no apparent loss due to adsorption or absorption of gacyclidine to the PTFE tubing.

### EXAMPLE 3

### Drug Compatibility Study with Polyurethane Catheters

Six 120 cm lengths of tubing containing thermoplastic polyurethane were filled with 350 μL of 3 mM gacyclidine in Ringer's solution (pH 5.5) and incubated at room temperature. Two tubing lengths were emptied for each time point (1 hr, 8 hr and 24 hr) and collected in an acid-washed autosampler vial. A 1:50 dilution in Ringer's solution was prepared from the collected samples. Concentrations of gacyclidine in the diluted samples were determined spectrophotometrically at 234 nm. There was an increase in gacyclidine concentration, presumably due to loss of water by evaporation through the tube walls. This was confirmed by a corresponding increase in solution osmolality as determined by use of a freezing point osmometer. The percentage increase in gacyclidine concentration corresponds quantitatively with the percentage increase in osmolality.

### EXAMPLE 4

### Drug Compatibility Study with Fluoropolymer-Lined Catheters

Fluoropolymer-lined (single-lumen) catheters were tested for drug compatibility with 100 μM gacyclidine in Ringer's Solution (pH 5.5). The lumens of the single-lumen catheters were filled with 200 μL of 100 μM gacyclidine in Ringer's solution and allowed to sit at room temperature. The ends of the devices were covered with paraffin wax vapor barrier to prevent evaporation. All three catheters were emptied after 48 hours into acid-washed HPLC autosampler vials. The concentration of gacyclidine was determined spectrophotometrically at 234 nm. The average overall percentage loss from experiments with three devices using 100 μM gacyclidine was 3.1% (1-5%, see Table 2).

TABLE 2
Average Concentration % Loss in Complete Devices

| | Gacyclidine | % Loss |
|---|---|---|
| Sample | Concentration (μM) | Total |
| Stock | 104.7 | 0 |
| 1 | 98.9 | 5.5 |
| 2 | 103.4 | 1.2 |
| 3 | 102.0 | 2.6 |
| Mean | 101.4 | 3.1 |

### Additional Embodiments

In some embodiments, materials used in fabricating electrodes should be chosen to have low affinity for drug substances, to not be thrombogenic, and to not promote stenosis. While titanium has low affinity for hydrophobic drugs, such as gacyclidine, titanium is known to be thrombogenic, as are steel, tungsten and platinum. As such, if titanium is employed to provide contact for electrical stimulation, it may optionally be positioned inside the catheter (as shown in FIGS. 17C and 17D) so as to avoid its thrombogenic effect when used in the presence of blood (for example resulting from surgical implantation of the catheter). This can be accomplished by placing the electrode in a recessed position within the catheter, such that contact with tissue and blood is reduced. Electrical connectivity between the electrode and tissue would then be maintained by ions present in the drug-containing vehicle (e.g., Ringer's solution), which also provides fluidic contact between the inside and outside of the catheter. Alternatively, a metal that is known to be non-thrombogenic, such as nickel (alone or as part of an alloy), could be used to provide electrical stimulation yet reduce the thrombogenicity of the electrode surface. Other less thrombogenic biocompatible materials can be used for electrodes such as Nitinol and titanium-aluminum-vanadium alloy.

Disorders of the middle and inner ear that can be treated by use of the drug delivery system described herein include: autoimmune inner ear disorder (AIED), Meniere's disease (idiopathic endolymphic hydrops), disorders of the inner ear associated with metabolic imbalances, infections, allergic or neurogenic factors, blast injury, noise-induced hearing loss, drug-induced hearing loss, tinnitus, presbycusis, barotrauma, otitis media (acute, chronic or serious), infectious mastoiditis, infectious myringitis, sensorineural hearing loss, conductive hearing loss, vestibular neuronitis, labyrinthitis, post-traumatic vertigo, perilymph fistula, cervical vertigo, ototoxicity, Mal de Debarquement Syndrome (MDDS), acoustic neuroma, migraine associated vertigo (MAV), benign paroxysmal positional vertigo (BPPV), eustachian tube dysfunction, cancers of the middle or inner ear, and bacterial, viral or fungal infections of the middle or inner ear. Cancers, bacterial, viral or fungal infections or endocrine, metabolic, neurological or immune disorders in other locations could also be treated by use of catheters similar in design to those described herein.

As previously indicated, devices similar to those described above for round window drug delivery can be sized or otherwise configured for placement into different regions of a patient's body for treating other conditions. For example, embodiments include catheters configured to deliver therapeutic substances to the vicinity of the auditory, optic, or other sensory nerves; to the eye, cochlea or other sensory organ for treating sensory disorders; to specific regions within the skin for local therapy; to the vicinity of the pituitary, adrenal, thymus, ovary, testis, or other gland for specific endocrine effects; to a region of the heart, pancreas, liver, spleen or other organ for organ-specific effects; and/or to specific regions of the brain or spinal cord for selective effects on the central nervous system. Embodiments also include methods employing such catheters, as well as methods employing catheters configured for round window drug delivery.

Numerous characteristics, advantages and embodiments of the invention have been described in detail in the foregoing description with reference to the accompanying drawings. However, the above description and drawings are illustrative only. The invention is not limited to the illustrated embodiments, and all embodiments of the invention need not necessarily achieve all of the advantages or purposes, or possess all characteristics, identified herein. Various changes and modifications may be effected by one skilled in the art without departing from the scope or spirit of the invention. Although example materials and dimensions have been provided, the invention is not limited to such materials or dimensions unless specifically required by the language of a claim. The elements and uses of the above-described embodiments can be rearranged and combined in manners other than specifically described above, with any and all permutations within the scope of the invention. As used herein (including the claims), “in fluid communication” means that fluid can flow from one component or region to another component or region; such flow may be by way of one or more intermediate (and not specifically mentioned) other components or region; and such flow may or may not be selectively interruptible (e.g., with a valve). As also used herein (including the claims), “coupled” includes two components that are attached (movably or fixedly) by one or more intermediate components.

1. A catheter, comprising:
a tubing portion having at least first and second lumens formed therein, wherein at least a portion of an internal surface of the first lumen is formed from a fluoropolymer; and
an end fitting coupled to a distal end of the tubing portion and having an internal fluid chamber and at least one fluid exit region, wherein
the first and second lumens are in fluid communication with the fluid chamber,
at least a portion of a surface of the fluid chamber is formed from a fluoropolymer, and
the fluid exit region is positioned so as to place the fluid chamber in fluid communication with a region external to the catheter.

2. The catheter of claim 1, wherein the end fitting is sized for placement in the round window niche of a human.

3. The catheter of claim 1, wherein the fluid exit region includes a plurality of holes.

4. The catheter of claim 1, wherein the end fitting includes an inflatable bladder coupled thereto, and wherein the tubing portion includes an additional lumen in fluid communication with an internal portion of the bladder.

5. The catheter of claim 1, wherein the end fitting includes a self-expanding ring coupled thereto.

6. The catheter of claim 5, further comprising a sheath, and wherein the tubing portion and end fitting fit within the sheath when the self-expanding ring is in a collapsed configuration, and wherein the catheter is configured such that the tubing portion can be pushed through the sheath so as to cause the end fitting to emerge from an end of the sheath.

7. The catheter of claim 1, further comprising:
an electrode located on a distal portion of the catheter, and
a wire placing the electrode in electrical communication with an electronics package configured to generate electrical stimulation pulses.

8. The catheter of claim 1, further comprising a pressure sensor coupled to the second lumen.

9. The catheter of claim 1, wherein the end fitting is removable, and wherein the catheter is configured to accept a second end fitting after said removal.

10. A catheter, comprising:
a tubing portion having at least first and second lumens formed therein, wherein at least a portion of an internal surface of the first lumen is formed from a fluoropolymer; and
a injection structure extending from a distal end of the tubing portion, the injection structure including first and second fluid passages, wherein
the first fluid passage is in fluid communication with the first lumen and has an opening configured to deliver fluid from the first lumen to a region external to the catheter, and
the second fluid passage is in fluid communication with the second lumen and has an opening configured to deliver fluid from the region external to the catheter to the second lumen.

11. The catheter of claim 10, further comprising a flexible insertion stop positioned on the end of the tubing portion.

12. The catheter of claim 10, wherein the first fluid passage opening is more distally located than the second fluid passage opening.

13. The catheter of claim 10, wherein the injection system comprises first and second needles, wherein the first fluid passage in within the first needle, and wherein the second fluid passage is within the second needle.

14. The catheter of claim 13, further comprising one or more electrical wires in electrical communication with an electronics package configured to generate electrical stimulation pulses to an ear tissue of a human when the end of the tubing portion is placed into a round window niche of a human.

15. A method, comprising:
placing an end fitting of a catheter into a round window niche of a human, wherein
the catheter includes a tubing portion having at least first and second lumens formed therein,
at least a portion of an internal surface of the first lumen is formed from a fluoropolymer,
the end fitting is coupled to a distal end of the tubing portion and includes an internal fluid chamber and at least one fluid exit region,
the first and second lumens are in fluid communication with the fluid chamber,
at least a portion of a surface of the fluid chamber is formed from a fluoropolymer, and
the fluid exit region is positioned so as to place the fluid chamber in fluid communication with a region external to the catheter;
delivering a drug-laden fluid through the first lumen to the internal fluid chamber; and
permitting excess drug-laden fluid to escape from the internal fluid chamber via the second lumen.

16. The method of claim 15, wherein the step of placing the end fitting of the catheter into a round window niche comprises placing the end fitting into the round window niche of a human having at least one of the following conditions: autoimmune inner ear disorder, Meniere's disease, a metabolic disorder, a bacterial infection, a viral infection, a fungal infection, an allergy, a neurological disorder, a blast injury, noise-induced hearing loss, drug-induced hearing loss, tinnitus, presbycusis, barotrauma, otitis media, infectious mastoiditis, infectious myringitis, sensorineural hearing loss, conductive hearing loss, vestibular neuronitis, labyrinthitis, post-traumatic vertigo, perilymph fistula, cervical vertigo, ototoxicity, Mal de Debarquement Syndrome, acoustic neuroma, migraine associated vertigo, benign paroxysmal positional vertigo, eustachian tube dysfunction, a malignant tumor, a non-malignant tumor, cancer of the middle ear, or cancer of the inner ear.

17. The method of claim 15, wherein the step of delivering a drug-laden fluid comprises delivering a fluid that includes at least one of gacyclidine, a neurologically active drug other than gacyclidine, an antibiotic, an anti-viral drug, an anti-inflammatory drug, an anti-cancer drug and a fungicide.

18. The method of claim 15, wherein an electrode is located on a distal portion of the catheter, and further comprising:
delivering electrical stimulation to ear tissue of the human through the electrode.

19. A method, comprising:
placing an end fitting of a catheter into a cochlea, an auditory nerve, an optic nerve, an eye, a pituitary gland, an adrenal gland, a thymus gland, an ovary, a testis, a heart, a pancreas, a liver, a spleen, a brain or a spinal cord of a human, wherein
the catheter includes a tubing portion having at least first and second lumens formed therein,
at least a portion of an internal surface of the first lumen is formed from a fluoropolymer,
the end fitting is coupled to a distal end of the tubing portion and includes an internal fluid chamber and at least one fluid exit region,
the first and second lumens are in fluid communication with the fluid chamber,
at least a portion of a surface of the fluid chamber is formed from a fluoropolymer, and
the fluid exit region is positioned so as to place the fluid chamber in fluid communication with a region external to the catheter;
delivering a drug-laden fluid through the first lumen to the internal fluid chamber; and
permitting excess drug-laden fluid to escape from the internal fluid chamber via the second lumen.

20. The method of claim 19, wherein the step of placing the end fitting of the catheter comprises placing the end fitting into a body region of a human having at least one of the following conditions: a metabolic disorder, a bacterial infection, a viral infection, a fungal infection, an allergy, a neurological disorder, or a cancer.

21. A method, comprising:
placing a distal end of a catheter into a round window niche of a human so as to pierce the round window membrane with first and second injection needles, wherein
the catheter includes a tubing portion having at least first and second lumens formed therein,
at least a portion of an internal surface of the first lumen is formed from a fluoropolymer,
the first injection needle extends from a distal end of the tubing portion,
an internal passage of the first injection needle is in fluid communication with the first lumen,
the second injection needle extends from the distal end of the tubing portion,
an internal passage of the second injection needle is in fluid communication with the second lumen, and
the catheter includes at least one wire in electrical communication with the first injection needle;
delivering a drug-laden fluid through the first lumen to the internal fluid chamber;
permitting excess drug-laden fluid to escape from the internal fluid chamber via the second lumen; and
delivering electrical stimulation to ear tissue of the human through the at least one wire.
